(19) United States
(12) Patent Application Publication
(10) Pub. No.: US 2021/0023244 A1
(43) Pub. Date: Jan. 28, 2021

(54) SUBLINGUAL AND BUCCAL ADMINISTRATIONS OF FLUORESCENT AGENTS FOR OPTICAL IMAGING

(71) Applicants: Board of Trustees of the Leland Stanford Jr Univer, Stanford, CA (US); The University of Connecticut, Farmington, CT (US)

(72) Inventors: Tulio A. Valdez, Palo Alto, CA (US); Xiuling Lu, Storrs, CT (US); Andre O'Reilly Beringhs, Sao Paulo (BR)

(21) Appl. No.: 17/035,490

(22) Filed: Sep. 28, 2020

Related U.S. Application Data
(63) Continuation of application No. PCT/US2019/025067, filed on Mar. 30, 2019.
(60) Provisional application No. 62/650,708, filed on Mar. 30, 2018.

(51) Int. Cl. A61K 49/00; A61K 9/00; A61K 47/36; A61K 47/44; A61K 47/10

(52) U.S. Cl. CPC A61K 49/0078; A61K 49/0034; A61K 9/0056; A61K 47/10; A61K 47/36; A61K 47/44; A61K 9/006

(57) ABSTRACT

Embodiments of fluorescent contrast agent compositions and formulations are provided for non-invasive administration, including oral, sublingual and buccal administration, e.g., for optical imaging of a subject's body part or tissue.

### RELATED APPLICATION DATA

The present application is a continuation of co-pending International Application No. PCT/US2019/025067, filed Mar. 30, 2019, which claims benefit of U.S. provisional application Ser. No. 62/650,708, filed Mar. 30, 2018, the entire disclosures of which are expressly incorporated by reference herein.

### FIELD OF THE INVENTION

The present invention relates generally to methods and agents for contrast enhancement during optical imaging, and, more particularly, to methods and agents that may provide substantially continuous contrast enhancement during optical imaging non-invasively, e.g., by sublingual or buccal use.

### BACKGROUND

Indocyanine green (“ICG”) dye is a fluorescent dye that may be administered intravenously to a patient for use as an indicator substance, e.g., for photometric hepatic function diagnostics or fluorescent angiography. ICG is typically available in powder form that is dissolved in a solvent for intravenous use.

Given that ICG is eliminated from the body with a half-life of about three to four minutes, ICG must be delivered continuously via an IV infusion or at higher (single or multiple) bolus doses, a requirement when undergoing an imaging procedure that may take substantial time. Further, some patients, e.g., pediatric patients, may not easily accept multiple injections, if necessary to complete a particular imaging procedure.

Accordingly, methods for administering fluorescent dyes, such as ICG, for various imaging and/or other medical procedures would be useful.

### SUMMARY

The present application describes various embodiments of fluorescent contrast agent compositions and formulations that may be provided for non-invasive administration, including oral, sublingual and buccal administration, e.g., for optical imaging of a subject's body part or tissue including imaging a subject's gastrointestinal tract or imaging to detect inflammation or infection in a subject.

The described embodiments and formulations may be used to replace currently available ICG solutions for intravenous administration, e.g., to identify cancerous lymph node metastases, to evaluate blood and lymphatic flow, or to detect or monitor inflammatory conditions in a subject. The described methods and formulations may provide a safer approach to optical imaging, particularly for children and the elderly, because they allow for the substitution of intravenously administered contrast agents and radioactive detection methods, and allow for an increased time of circulation of ICG or other near infrared fluorescent dyes, e.g., IRDye® 800CW made by Li-Cor Biosciences, by providing continuous release of the contrast dyes.

Some embodiments are chitosan-based sublingual formulations (films') including ICG or IRDye® 800CW as fluorescent probes, which may facilitate prompt and/or steady release of the dye, e.g., in the form of an emulsion or microemulsion. Other embodiments are chitosan-based, self-emulsifying films including ingredients such as castor oil, polysorbate 80 (e.g., Tween® 80), polyoxyl castor oil (e.g., Kolliphor® RH 40), PEG 400, and ICG or IRDye® 800CW. The embodiments may exhibit good flexibility and apparent stickiness, making them adequate for sublingual and buccal administration.

In accordance with one embodiment, a method is provided for enhancing contrast of an image of a body part or tissue that includes orally administering fluorescent dye that is formulated for substantially continuous release. For example, a film or other substrate carrying the dye may be administered orally, e.g., buccally or sublingually, and allowed to dissolve over an extended period of the time to substantially continuously release the dye.

In accordance with another embodiment, a device is provided for orally administering fluorescent dye to a patient that includes bioabsorbable base material, and fluorescent dye carried by the base material such that the fluorescent dye is released substantially continuously for an extended period of time, e.g., at least thirty minutes, when administered orally to the patient. For example, the device may be formed as a film sized for oral administration.

In accordance with yet another embodiment, a method is provided for making an agent for oral administration to a patient to enhance optical imaging that includes mixing a solution of bioabsorbable base material; adding fluorescent dye to the solution in a predetermined concentration; pouring the solution into a container; and drying the solution in the container to provide a film. Alternatively, the films may be produced using a substantially continuous and/or other high-throughput process. The film may be separated into individual doses for subsequent administration to individual patients or subjects.

Other aspects and features including the need for and use of the present invention will become apparent from consideration of the following description taken in conjunction with the accompanying drawings.

### BRIEF DESCRIPTION OF THE DRAWINGS

The invention is best understood from the following detailed description when read in conjunction with the accompanying drawings. It is emphasized that, according to common practice, the various features and design elements of the drawings are not to-scale. On the contrary, the dimensions of the various features and design elements are arbitrarily expanded or reduced for clarity. Included in the drawings are the following figures.

FIG. 1 shows an example of a bioabsorbable film including fluorescent dye. The dye may be present as particulate material, as shown, or as a molecular dispersion, where particles cannot be visually observed under naked eye.

FIG. 2 shows the chemical structure of Chitosan.

FIGS. 3A and 3B show representative in vivo optical imaging data after dosing mice using ICG films.

FIG. 4 is a graph showing exemplary fluorescence signals quantified on mice's back paws versus time after sublingual dosing with ICG-loaded films. The red curve (circle points) refers to regular ICG film and the blue curve (square points) refers to self-emulsifying ICG film.

FIG. 5 is a graph showing exemplary fluorescence signals quantified on mice's back paws versus time after sublingual dosing with IRDye® 800CW films. The red curve (circle points) refers to regular IRDye® 800CW film and the blue curve (square points) refers to self-emulsifying IRDye® 800CW film. The signal quantified on the mice's back paws refers to the bioavailable dye, which can be detected in vascular circulation due to the high density of superficial blood vessels in this anatomical region of the animals.

FIG. 6 is a graph showing the in vitro release kinetics of ICG in aqueous media at physiological temperature by pre-attaching the films to the bottom of dissolution vessels (USP II apparatus).

### DETAILED DESCRIPTION OF THE EXEMPLARY EMBODIMENTS

Before the exemplary embodiments are described, it is to be understood that the invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials are now described.

It must be noted that as used herein and in the appended claims, the singular forms “a,” “an,” and “the” include plural referents unless the context clearly dictates otherwise. Thus, for example, reference to “a compound” includes a plurality of such compounds and reference to “the polymer” includes reference to one or more polymers and equivalents thereof known to those skilled in the art, and so forth.

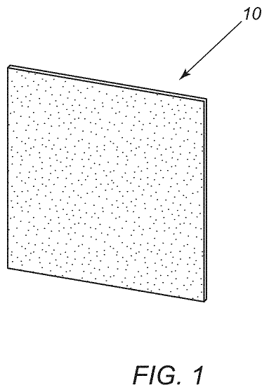

Turning the drawings, FIG. 1 shows an example of a device for orally administering fluorescent dye in the form of a film 10. Generally, the film 10 is formulated from a bioabsorbable carrier, substrate, or other base material and fluorescent dye, e.g., indocyanine green (“ICG”) or near infrared fluorescent due (e.g., IRDye® 800CW). The dye may be included in the formulation in the free molecular form or complexed with other components to allow for self-emulsification in contact with aqueous media. The bioabsorbable carrier along with the self-emulsifying components may be denominated “platform matrix” and it can carry a variety of fluorescent dyes. In vivo, the dye may be released from the film in its free molecular form and/or as a “complex” in the form of microemulsion droplets.

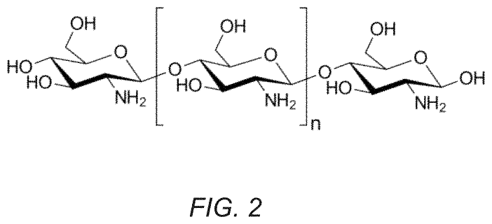

In an exemplary embodiment, the base material may be a bioabsorbable polymer, e.g., chitosan, e.g., as shown in FIG. 2. Chitosan is a linear polysaccharide composed of randomly distributed β-(1→4)-linked D-glucosamine (deacetylated unit) and N-acetyl-D-glucosamine (acetylated unit). This natural polymer may be useful as a film-forming polymer due to its known mucoadhesive properties, which are derived from its positive charge (amine groups) that interact with negatively charged mucin proteins present in mucus. It will be appreciated that other bioabsorbable polymers may be used that are biocompatible and dissolve over an extended time, e.g., to provide substantially continuous release of the dye, as described elsewhere herein.

Alternatively, the film 10 may be formulated as a self-emulsifying film, e.g., forming a microemulsion as it dissolves, including an oil, a surfactant, a co-surfactant, and, optionally, a co-solvent. For example, a mixture including castor oil, polysorbate 80 (e.g., Tween® 80), polyoxyl castor oil (e.g., Kolliphor® RH40), and polyethylene glycol may be used to form the base material, e.g., using the exemplary materials and methods described elsewhere herein.

Optionally, the film 10 may include other ingredients, e.g., flavor or taste-masking materials, visible dyes, and the like, e.g., to make the film 10 more palatable for oral administration.

The film 10 (or other substrate) may be provided in a size appropriate for oral administration, e.g., for buccal or sublingual administration, for example, in rectangular, square, circular, or other shapes having a width, diameter, or other maximum dimension, e.g., not more than about five centimeters, or not more than about two centimeters. The film 10 may have a substantially uniform thickness, e.g., not more than about five millimeters (5 mm), e.g., to facilitate sublingual or buccal placement.

The film 10 may be formulated from a solution of the base material and the dye such that the dye is dispersed or dissolved substantially uniformly through the base material, e.g., to provide substantially uniform and/or steady rate of delivery during administration. The rate of release may be configured in a predetermined manner by appropriately formulating the base material, e.g., to provide a substantially fixed release rate, to provide an initially increased release rate, a tapered release rate, and the like.

Alternatively, the base material and dye may be provided in other constructions, e.g., a multiple layer film, a tablet, and the like (not shown). For example, a multiple layer film may be formed that includes an internal reservoir containing the dye, and the base material may be porous or dissolve in a predetermined manner to release the dye at a desired rate from the reservoir.

Specific examples of methods for making an agent for oral administration are described below. Generally, however, the method includes mixing a solution of bioabsorbable base material, e.g., chitosan or other bioabsorbable polymer; adding fluorescent dye, optionally including a self-emulsifying drug delivery system (“SEDDS”), to the solution in a predetermined concentration; pouring the solution into a container, e.g., a flat dish; and drying the solution in the container to provide a film or other solid structure. Alternatively, the films may be produced using a substantially continuous and/or other high-throughput process, yielding an equivalent product. The films may result in a desired concentration of dye, e.g., not more than about fifty percent (50%) dye weight to film weight, not more than twenty five percent (25%), not more than ten percent (10%), not more than five percent (5%), and the like. The film/structure may be separated into individual doses for subsequent administration to individual patients. In exemplary embodiments, each dose may include a desired amount of dye based on the duration of the intended procedure and/or size of the patient, e.g., between about one and fifty milligrams per kilogram (1.0-50 mg/kg).

During use, a film 10 (or other form) including an appropriate dose of the fluorescent dye, e.g., between about 0.01-15% (w/w) of the weight of the film, may be selected and administered to the patient or other subject orally, e.g., buccally or sublingually. The film 10 may automatically adhere to the sublingual mucosa, e.g., due to the adhesive properties of the chitosan or other base material. Alternatively, the film 10 may be moistened immediately before administration, e.g., to enhance adhesion such that the film 10 does not migrate within the patient's mouth during the imaging procedure. The dye may be released from the film 10 and absorbed by the mucosa to deliver the dye into the patient's vasculature, which may deliver the dye throughout the patient's body, including a target body part or tissue region, which may be imaged using optical imaging. In addition or alternatively, the dye may be travel from the patient's mouth into their gastrointestinal system, e.g., for imaging the upper gastrointestinal system, and/or to pass through the gastrointestinal system and enter the vascular system, although the resulting dosage may be lower than direct absorption through the mucosa.

The film 10 may dissolve over an extended period of time, e.g., at least about thirty minutes, at least one hour, at least two hours, and the like, e.g., extending up to twenty four (24) hours, thereby providing a steady release of the dye to allow an optical imaging and/or other procedure to be performed on one or more target body parts or tissue regions, target locations within the gastrointestinal system, and the like.

In exemplary embodiments, the film 10 (or other form) may be used to orally administer fluorescent dye for optical imaging procedures to identify cancerous lymph node metastases, to evaluate blood and lymphatic flow, or to detect or monitor inflammatory conditions in a subject. Such films may provide a safer approach to optical imaging, particularly for children and the elderly, because they allow for the substitution of intravenously administered contrast agents and radioactive detection methods, providing increased comfort to the patient, and/or allow for an increased time of circulation of ICG and IRDye® 800CW by providing continuous release of the contrast dyes.

Various examples of formulations will now be described.

### EXAMPLE 1: PREPARATION OF INDOCYANINE GREEN (ICG) AND IRDYE® 800CW SUBLINGUAL FILMS

This example includes exemplary methods for making films carrying fluorescent dye. Generally, chitosan solution was prepared at a 1% w/w concentration in 1% acetic acid solution (w/v) and stored at 4° C. until used in each of the examples.

Indocyanine Green Regular Film (2.4% w/w)

1. Chitosan 1% (w/w) solution was transferred into an Unguator® jar.

2. ICG powder was added into the same Unguator® jar to give a 2.4% (w/w) concentration with respect to the solid content (dye, film-forming base and other non-volatile components).

3. The formulation was homogenized using an Unguator® e/s system.

4. Six grams (6 g) of the homogenized formulation were poured onto a circular plastic dish.

5. The formulation was left to dry and form a thin film for twenty four (24) hours in an air-circulating oven at 40° C.

Indocyanine Green Self-Emulsifying (“SE”) Film (2.4% w/w)

1. Self-emulsifying drug delivery systems (“SEDDS”) containing ICG were prepared by mixing the following components in a glass vial:

| | Concentration |
|---|---|
| Component | (% w/w) |
| Castor oil | 20.0 |
| Tween ® 80 | 20.0 |
| Kolliphor ® RH40 | 35.0 |
| Polyethylene glycol 400 | 25.0 |
| Total | 100 |

2. The resulting dispersion was stirred for three hours at 60° C. until a clear solution was obtained. ICG was loaded onto the blank SEDDS at a 30 mg/g concentration.

3. ICG-loaded SEDDS and chitosan solution were mixed in an Unguator® jar to achieve a final ICG concentration of 2.4% (w/w) with respect to the solid content (dye, film-forming base and other non-volatile components).

4. The formulation was homogenized using an Unguator® e/s system.

5. Six grams (6 g) of the homogenized formulation were poured onto a circular plastic dish.

6. The formulation was left to dry and form a thin film for twenty four (24) hours in an air-circulating oven at 40° C.

IRDye® 800CW Regular Film (1% w/w)

1. Chitosan 1% (w/w) solution was transferred into an Unguator® jar.

2. IRDye® 800 CW was added into the same Unguator® jar to give a 1.0% (w/w) concentration with respect to the solid content (dye, film-forming base and other non-volatile components).

3. The formulation was homogenized using an Unguator® e/s system.

4. Six grams (6 g) of the homogenized formulation were poured onto a circular plastic dish.

5. The formulation was left to dry and form a thin film for twenty four (24) hours in an air-circulating oven at 40° C.

IRDye® 800CW Self-Emulsifying (“SE”) Film (1% w/w)

1. Self-emulsifying drug delivery systems (“SEDDS”) containing ICG were prepared by mixing the following components in a glass vial:

| | Concentration |
|---|---|
| Component | (% w/w) |
| Castor oil | 20.0 |
| Tween ® 80 | 20.0 |
| Kolliphor ® RH40 | 35.0 |
| Polyethylene glycol 400 | 25.0 |
| Total | 100 |

2. The resulting dispersion was stirred for three hours at 60° C. until a clear solution was obtained. IRDye® 800CW was loaded onto the blank SEDDS at a 30 mg/g concentration.

3. IRDye® 800CW-loaded SEDDS and chitosan solution were mixed in an Unguator® jar to achieve a final IRDye® 800 CW concentration of 1.0% (w/w) with respect to the solid content (dye, film-forming base and other non-volatile components).

4. The formulation was homogenized using an Unguator® e/s system.

5. Six grams (6 g) of the homogenized formulation were poured onto a dish (35 mm diameter, 38.48 cm² area).

6. The formulation was left to dry and form a thin film for twenty four (24) hours in an air-circulating oven at 40° C.

The resulting structures were cut into square shapes, e.g., similar to the film 10 shown in FIG. 1, to provide sublingual films for animal dosing.

Examples of in vivo tests that were performed will now be described.

### EXAMPLE 2: IN VIVO SUBLINGUAL DOSING OF INDOCYANINE GREEN (“ICG”) AND IRDYE® 800 CW FILMS

In Vivo Sublingual Administration of ICG Films for Upper Gastrointestinal Tract Imaging

1. In this example, ICG films, both regular and self-emulsifying, were cut into small squares to give a total ICG dose of 50 μg/mouse.

2. Films were pre-wetted in PBS pH 7.2 and directly applied to the sublingual space in the mice's buccal cavities (n=3 per treatment group), which were under anesthesia during the procedure (1.5 liter/min O₂, 2.0% isoflurane).

3. Animals were kept under anesthesia for thirty min (1.5 liter/min O₂, 2.0% isoflurane) to ensure adequate adhesion of the films to the sublingual mucosa, and imaging was performed afterwards using an in vivo optical imaging system.

4. Images were collected before sublingual dosing and at 0.5, 1, 3, 6, 12 and 24 hours post-dosing using an in vivo optical imaging system, focusing on the head and neck area of the animals.

In Vivo Sublingual Administration of ICG Films for Back Paw Imaging

1. ICG films, both regular and self-emulsifying, were cut into small squares to give a total ICG dose of 50 μg/mouse.

2. Films were pre-wetted in PBS pH 7.2 and directly applied to the sublingual space in the mice's buccal cavities (n=3 per treatment group), which were under anesthesia during the procedure (1.5 liter/min O₂, 2.0% isoflurane).

3. Animals were kept under anesthesia for thirty min (1.5 liter/min O₂, 2.0% isoflurane) to ensure adequate adhesion of the films to the sublingual mucosa.

4. Images were collected before sublingual dosing and at 0.5, 1, 3, 6, 12 and 24 hours post-dosing using an in vivo optical imaging system, focusing on the right-flank area of the animals.

In Vivo Sublingual Administration of IRDye® 800CW Films for Back Paw Imaging

1. IRDye® 800CW films, both regular and self-emulsifying, were cut into small squares to give a total IRDye® 800CW dose of 25 μg/mouse.

2. Films were pre-wetted in PBS pH 7.2 and directly applied to the sublingual space in the mice's buccal cavities (n=3 per treatment group), which were under anesthesia during the procedure (1.5 liter/min O₂, 2.0% isoflurane).

3. The animals were kept under anesthesia for thirty min (1.5 liter/min O₂, 2.0% isoflurane) to ensure adequate adhesion of the films to the sublingual mucosa.

4. Images were collected before sublingual dosing and at 0.5, 1, 3, 6, 12 and 24 hours post-dosing using an in vivo optical imaging system, focusing on the right-flank area of the animals.

### EXAMPLE 3: IN VIVO UPPER GASTROINTESTINAL TRACT AND BACK PAW OPTICAL IMAGING FOLLOWING SUBLINGUAL ADMINISTRATION OF ICG FILMS

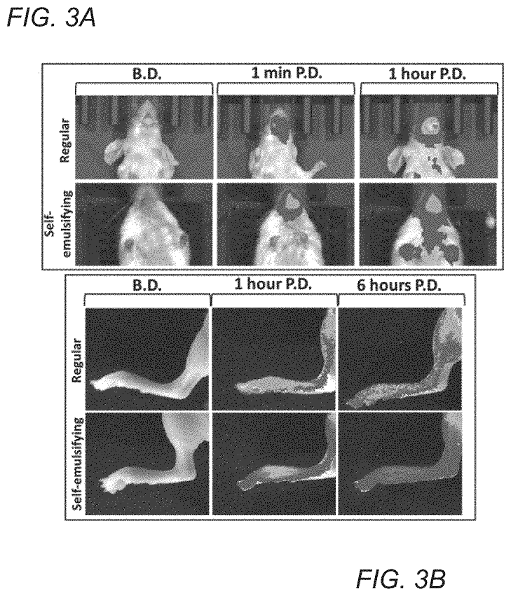

In this example, nude mice were treated with ICG-loaded regular and self-emulsifying films (n=3). The sublingual administration was performed under anesthesia to allow better adhesion of the films to the sublingual mucosa. The model films were tested using in vivo fluorescence imaging post sublingual dosing on mice, as shown in the representative images in FIGS. 3A-3C. Excellent upper gastrointestinal tract imaging ability was observed for the self-emulsifying film. The films hydrated fast, and ICG was steadily released in the form of an emulsion. Due to the high concentration of dye released in the buccal cavity, the mice's saliva was rich in ICG. As the saliva was swallowed, the dye's track as it moved through the upper GI tract could be easily imaged using non-invasive non-radioactive fluorescence imaging techniques. As early as one-hour post sublingual dosing, the dye was easily detected moving down the trachea of the animal, giving excellent and continuous contrast for applications such as swallowing evaluation. The regular film, on the other hand, released ICG in a more conservative way, therefore it was less efficient for GI tract imaging, being retained mainly in the buccal cavity of the animal.

For example, FIG. 3A shows in vivo fluorescence imaging of mice post sublingual dosing of ICG films, focusing on the imaging ability of the upper gastrointestinal tract, FIG. 3B shows in vivo fluorescence imaging of mice paws post sublingual dosing of ICG films, indicating the systemic absorption of the dye post dosing. As used in these drawings, “B.D.” means before dosing, and “P.D.” means post dosing.

Besides gastrointestinal tract imaging, ICG films also provide a non-invasive imaging tool for infection/inflammation diagnosis. To accomplish this, ICG has to be absorbed and become bioavailable in the blood stream to reach these sites of interest. By using fluorescence imaging on the back paws of the animals treated with ICG films, an increase in fluorescence signal was detected, e.g., as shown in FIG. 3B. Since the paws are rich in blood vessels, such increase in fluorescence correlates directly to the amount of dye circulating in the blood stream. In this case, it was verified that the regular ICG film outperforms the self-emulsifying one. Since this film releases the dye at a slower rate, and it remains in the sublingual compartment, the dye is able to permeate the thin sublingual membrane and reach the blood stream faster and at a higher concentration compared with the self-emulsifying film. For the second one, the dye self-emulsifies, and it is swallowed alongside the animal's saliva. After it reaches the stomach and intestines, bioavailability and the paw signal are lower due to the metabolic loss from first-pass liver metabolism.

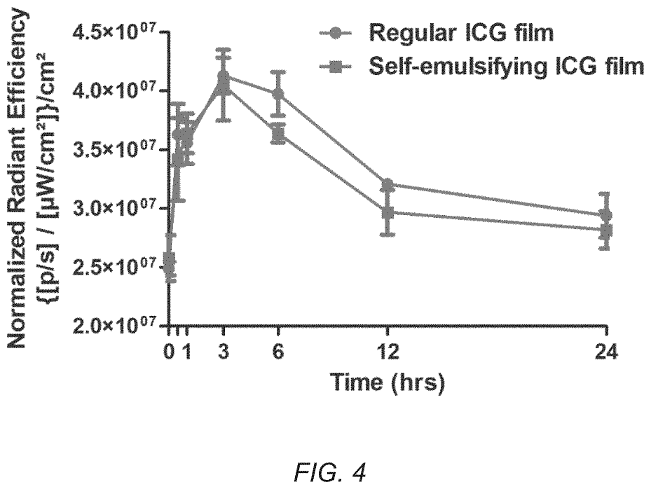

FIG. 4 displays pharmacokinetic curves generated based on the fluorescence signal quantified on the paw of the animals. As can be seen, the area under the curve of regular ICG film is higher compared to self-emulsifying film. This indicates a higher systemic exposure of the body to the dye when administered via regular ICG sublingual film. In general, maximum dye concentration in plasma is achieved roughly three (3) hours post dosing. The signal remains above background for up to twelve (12) hours, demonstrating one of the advantages of using these films for sustained delivery of ICG over a prolonged period of time.

### EXAMPLE 4: IN VIVO BACK PAW OPTICAL IMAGING FOLLOWING SUBLINGUAL ADMINISTRATION OF IRDYE® 800CW FILMS

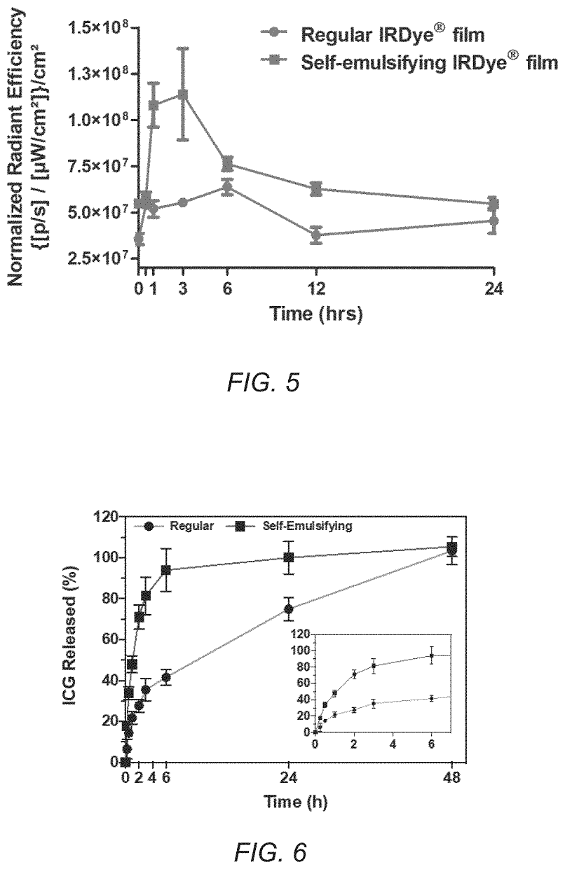

In this example, nude mice were treated with IRDye® 800CW films via sublingual route (both regular and self-emulsifying), and fluorescence imaging was performed at various time points (n=3). FIG. 5 displays pharmacokinetic curves generated based on the fluorescence signal quantified on the paw of the animals. Similar to ICG film, we can clearly see that the area under the curve of regular IRDye® 800CW film is higher compared with the self-emulsifying film.

### EXAMPLE 5: IN VITRO RELEASE KINETICS OF INDOCYANINE GREEN-LOADED FILMS

Regular and self-emulsifying films containing ICG were assessed for their in vitro release kinetics by pre-attaching the films to the bottom of dissolution vessels in a United States Pharmacopeia (USP) apparatus II (paddle method). Release studies were performed using water at 37° C. as release medium.

Regular films released ICG slowly when compared with the self-emulsifying films. Regular films showed 21.75±3.04% release within the first hour of the assay, whereas self-emulsifying films showed a 47.91±4.22%. These findings are reasonable considering the semi-solid properties of the self-emulsifying film, as well as the ability to foster the release of ICG in the form of microemulsion.

The described formulations have the potential of modulating the release kinetics of ICG and other dyes of interest to a wide variety of release rates and extents. The composition and properties of the formulations (e.g. polymer concentration, polymer to dye ratio, film thickness, self-emulsifying components) can be changed in order to tailor the release kinetics to different needs. For instance, for upper gastrointestinal imaging, faster release kinetics are desired. However, for systemic absorption, slower release kinetics may be beneficial due to the limiting absorption capacity of the sublingual space.

Further, in describing representative embodiments, the specification may have presented the method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the scope of the appended claims.

1. A method for enhancing contrast of an image of a body part or tissue, comprising orally administering fluorescent dye that is formulated for continuous release.

2. The method of claim 1, wherein the dye is formulated in a platform matrix and released as free dye or as a complex.

3. The method of claim 2, wherein the platform matrix is a film.

4. The method of claim 2, wherein the complex is a microemulsion.

5. The method of claim 1, wherein the administration is sublingual.

6. The method of claim 1, wherein the administration is buccal.

7. The method of claim 1, wherein the fluorescent dye is a near infrared fluorescent dye.

8. The method of claim 7, wherein the near infrared fluorescent dye is indocyanine green and its derivatives.

9. The method of claim 1, wherein the dye is used for imaging and diagnosis.

10. The method of claim 1, further comprising acquiring one or more images of the body part or tissue.

11. A device for administering fluorescent dye to a patient, comprising:
bioabsorbable base material; and
fluorescent dye carried by the base material such that the fluorescent dye is released substantially continuously for an extended period of time when administered orally to the patient.

12. The device of claim 11, wherein the base material is formed as a film sized for oral administration.

13. The device of claim 11, wherein the dye and base material are formulated as a platform matrix that releases the dye as free dye.

14. The device of claim 11, wherein the dye and base material are formulated as a complex.

15. The device of claim 14, wherein the complex is a microemulsion.

16. The device of claim 11, wherein the base material comprises chitosan.

17. The device of claim 11, wherein the dye comprises ingredients to form a self-emulsifying composition.

18. The device of claim 17, wherein the ingredients comprise an oil, a surfactant, and a co-surfactant.

19. The device of claim 17, wherein the ingredients comprise castor oil, polysorbate 80, polyoxyl castor oil, and polyethylene glycol.

20-21. (canceled)

22. A method for making an agent for oral administration to a patient to enhance optical imaging, comprising:
mixing a solution of bioabsorbable base material;
adding fluorescent dye to the solution in a predetermined concentration;
pouring the solution into a container; and
drying the solution in the container to provide a film.

23-29. (canceled)
